(19) United States
(12) Patent Application Publication
(10) Pub. No.: US 2025/0000911 A1
(43) Pub. Date: Jan. 2, 2025

(54) Autophagosome For Promoting Healing of Diabetic Wound, and Preparation Method and Use Thereof

(71) Applicant: CHINESE PLA GENERAL HOSPITAL, Beijing (CN)

(72) Inventors: Cuiping Zhang, Beijing (CN); Shengnan Cui, Beijing (CN); Xi Liu, Beijing (CN); Kui Ma, Beijing (CN); Xiaobing Fu, Beijing (CN)

(21) Appl. No.: 18/603,959

(22) Filed: Mar. 13, 2024

(30) Foreign Application Priority Data
Jun. 29, 2023 (CN) 202310779646.3

(51) Int. Cl. A61K 35/44; A61P 17/02; C12N 5/071

(52) U.S. Cl. CPC A61K 35/44; A61P 17/02; C12N 5/069

(57) ABSTRACT

The present disclosure discloses an autophagosome for promoting healing of a diabetic wound, and a preparation method and use thereof. In the preparation method of the autophagosome provided in the present disclosure, the autophagosome is extracted from a starved vascular endothelial cell, and the production of the autophagosome does not require induction by an additional reagent. The preparation method of the present disclosure involves simple extraction conditions, does not require an ultracentrifuge, and can be implemented in an ordinary laboratory. In addition, a yield of the autophagosome is higher than a yield of an exosome. The present disclosure provides an easy-to-implement and low-cost treatment method without toxic and side effects for a patient suffering from a chronic refractory wound. The autophagosome of the present disclosure can promote healing of a diabetic wound, and reduce physiological and psychological burdens caused by a diabetic wound to a patient.

### CROSS REFERENCE TO RELATED APPLICATION

This patent application claims the benefit and priority of Chinese Patent Application No. 2023107796463, filed with the China National Intellectual Property Administration on Jun. 29, 2023, the disclosure of which is incorporated by reference herein in its entirety as part of the present application.

### TECHNICAL FIELD

The present disclosure belongs to the technical field of biomedicine, and specifically relates to an autophagosome for promoting healing of a diabetic wound, and a preparation method and use thereof.

### BACKGROUND

Diabetes is a multifaceted metabolic disorder involving biochemical disturbances and epigenetic factors, which are ultimately converted into an irreversible tissue change in a glucose oxidation process. Leg or foot ulcers are the most common complications in diabetic patients, and about 19% to 34% of diabetic patients suffer from leg or foot ulcers. Diabetic foot is classically defined as a deep tissue injury to lower limbs, and is also called a “cancer analogy” by Armstrong, which is mainly due to the fact that a 5-year mortality rate of foot ulcers and amputations exceeds a 5-year mortality rate of common cancers. Globally, about 50% to 70% of amputations are caused by diabetic wounds. It is generally believed that major features of diabetic wounds include persistent infection and impaired generation and remodeling of mature granulation tissues. Although some progress has been made for the understanding of impaired healing of diabetic wounds in recent years, pathological and molecular mechanisms of impaired healing of diabetic wounds are still unclear, and existing methods exhibit unsatisfactory therapeutic effects.

Secretory autophagosomes (SAPs) are produced through secretory autophagy, and belong to the family of extracellular vesicles (EVs). SAP is a novel mode for cell-cell communication by transferring a cargo to a target cell. The loss of integrity of a lysosome can be induced under a stress and particularly under starvation to enhance secretory autophagy and reduce degradative autophagy. In recent years, the research on SAPs has mainly focused on elucidating roles of SAPs in the occurrence and progression of diseases such as cancer and acute respiratory distress syndrome, but therapeutic effects of SAPs in diseases has been neglected.

### SUMMARY

An objective of the present disclosure is to provide an autophagosome for promoting healing of a diabetic wound, and a preparation method and use thereof. The autophagosome can improve a healing effect of a diabetic wound.

The present disclosure provides a preparation method of an autophagosome for promoting healing of a diabetic wound, including the following steps: cultivating a vascular endothelial cell in a serum-free medium; subjecting a resulting cell culture to first centrifugation to obtain a first supernatant, and collecting the first supernatant; and subjecting the first supernatant to second centrifugation to obtain a precipitate including the autophagosome,
where the first centrifugation is conducted at a centrifugal force of 2,000 g for 10 min; and
the second centrifugation is conducted at a centrifugal force of 12,000 g for 15 min.

Preferably, the serum-free medium includes a 0.1% penicillin-streptomycin (PS)-containing Gibco high-glucose Dulbecco's Modified Eagle Medium (DMEM).

Preferably, the vascular endothelial cell is cultivated in the serum-free medium for 48 h.

Preferably, the first centrifugation and the second centrifugation both are conducted at 4° C.

Preferably, after the precipitate is collected, the preparation method further includes incubating the precipitate with an LC3-labeled magnetic bead to capture the autophagosome.

The present disclosure also provides an autophagosome prepared by the preparation method described above.

The present disclosure also provides a use of the autophagosome described above in preparation of a drug for promoting healing of a diabetic wound.

Beneficial effects: The present disclosure provides a preparation method of an autophagosome for promoting healing of a diabetic wound. In the preparation method, the autophagosome is extracted from a starved vascular endothelial cell, and the production of the autophagosome does not require induction by an additional reagent. The preparation method involves simple extraction conditions, does not require an ultracentrifuge, and can be implemented in an ordinary laboratory. In addition, a yield of the autophagosome is higher than a yield of an exosome. The present disclosure provides an easy-to-implement and low-cost treatment method without toxic and side effects for a patient suffering from a chronic refractory wound. The autophagosome of the present disclosure can promote healing of a diabetic wound, and reduce physiological and psychological burdens caused by a diabetic wound to a patient.

### BRIEF DESCRIPTION OF THE DRAWINGS

FIG. 1 shows an expression level of an LC3 protein in a vascular endothelial cell and an autophagosome extracted from the vascular endothelial cell in Example 2 of the present disclosure and a corresponding nanoparticle analysis result;

FIG. 2 shows a nanoparticle analysis result of an autophagosome in Example 2 of the present disclosure;

FIG. 3 shows transmission electron microscopy images of an autophagosome in Example 2 of the present disclosure;

FIGS. 4A-C show general wounds of diabetic mice treated with an autophagosome in Example 2 of the present disclosure; and

FIGS. 5A-B show healing trajectory of a wound of a diabetic mouse treated with an autophagosome in Example 2 of the present disclosure.

### DETAILED DESCRIPTION OF THE EMBODIMENTS

The present disclosure provides a preparation method of an autophagosome for promoting healing of a diabetic wound, including the following steps: a vascular endothelial cell is cultivated in a serum-free medium; a resulting cell culture is subjected to first centrifugation to obtain a first supernatant, and the first supernatant is collected; and the first supernatant is subjected to second centrifugation to obtain a precipitate including the autophagosome,
where the first centrifugation is conducted at a centrifugal force of 2,000 g for 10 min; and
the second centrifugation is conducted at a centrifugal force of 12,000 g for 15 min.

In the present disclosure, the vascular endothelial cell is cultivated with the serum-free medium, and the serum-free medium is preferably a 0.1% PS-containing Gibco DMEM. In the present disclosure, the vascular endothelial cell is preferably cultivated in the serum-free medium for 48 h. In the present disclosure, the serum-free medium is used without fetal bovine serum to make the vascular endothelial cell in a starvation state, thereby promoting the production of SAPs.

In the present disclosure, the cell culture is preferably subjected to the first centrifugation. The first centrifugation includes: low-speed centrifugation is conducted to remove large-particle matters, and then the first supernatant is collected. In the present disclosure, the first centrifugation is conducted at a low temperature, such as 4° C. In the present disclosure, the first supernatant is subjected to second centrifugation, and then the precipitate is collected. The second centrifugation is also conducted at a low temperature. In the present disclosure, the second centrifugation is preferably conducted twice, where a resulting precipitate is resuspended with phosphate buffered saline (PBS), and then a resulting suspension is subjected to high-speed centrifugation to obtain a crude autophagosome.

In the present disclosure, after the precipitate is collected, the preparation method preferably further includes: the precipitate is incubated with an LC3-labeled magnetic bead to capture the autophagosome. In an embodiment, preferably, an activated protein A magnetic bead is first co-incubated with an LC3 antibody, then collected through magnetic separation, co-incubated with the crude autophagosome collected above at 37° C. for 30 min to 2 h, and then subjected to elution with 0.1 M NaOH.

The present disclosure also provides an autophagosome prepared by the preparation method described above.

The purified autophagosome obtained by the preparation method has a classic double-layer membrane-like structure, is in a cup-like shape overall, and has a particle size of (415.8±69 nm) and a concentration of (2.2×10¹¹). In addition, an expression level of LC3II in the autophagosome is significantly higher than an expression level of LC3II in a cell lysate, while an expression level of LC3I in the autophagosome is significantly reduced, indicating that the extracted EV is an exosome.

The present disclosure also provides a use of the autophagosome described above in preparation of a drug for promoting healing of a diabetic wound. The use of the autophagosome can significantly promote healing and repair of a wound, epithelialization, and regeneration of a granulation tissue in diabetic mice, indicating that the autophagosome has a great application potential in stimulation of regeneration of skin appendages and improvement of a healing quality.

In order to further illustrate the present disclosure, the autophagosome for promoting healing of a diabetic wound and the preparation method and use thereof provided by the present disclosure are described in detail below with reference to the accompanying drawings and examples, but the accompanying drawings and the examples should not be construed as limiting the protection scope of the present disclosure.

### EXAMPLE 1

### Preparation of an Autophagosome for Promoting Healing of a Diabetic Wound

1. Gibco high-glucose DMEM including only 5% PS was set as a starvation environment.
2. A vascular endothelial cell was cultivated in a serum-free environment for 48 h, and then a resulting cell culture was collected and centrifuged at 2,000 rpm for 10 min in a centrifuge to remove large-particle matters (dead cells and debris) to obtain a first precipitate and a first supernatant; and the first supernatant was collected and further centrifuged (4° C., 12,000 g, 15 min) to obtain a second precipitate and a second supernatant, the second supernatant was discarded, and the second precipitate was resuspended with PBS and then centrifuged (4° C., 12,000 g, 15 min) to obtain a crude autophagosome.
3. Then, a protein A magnetic bead was co-incubated with an LC3 antibody, then collected through magnetic separation, and co-incubated with the crude autophagosome collected above to purify the autophagosome through an antigen-antibody interaction.
4. A resulting precipitate was resuspended with sterile PBS to obtain an autophagosome solution, which could be used immediately or frozen for later use.

### EXAMPLE 2

In this example, based on the method in Example 1, relevant verifications were conducted according to an obtained exosome, including the following steps:
1. After a total protein was extracted from an autophagosome, a protein concentration was detected by a BCA protein assay kit, and a surface-specific marker LC3 of the autophagosome was detected by a western blot experiment, including electrophoresis, transfer, blocking, primary and secondary antibody incubation, and chemiluminescence imaging analysis. Specific operations were as follows:
(1) Detection of a protein concentration by a BCA method: About 50 μL of an autophagosome suspension was taken and added to 50 μL of an RIPA lysis buffer (R0010, Solarbio) to allow full lysis at 4° C. for 15 min; a resulting lysate was centrifuged (12,000 g, 4° C.) for 20 min (100 μL of the RIPA lysis buffer was adopted for cells in each 6-well plate), and a resulting supernatant was slowly transferred by a pipette to a 1.5 mL EP tube; a BCA protein concentration assay kit (PC0020, Solarbio) was used to determine a protein concentration in a cell lysate and an autophagosome, and two groups of samples each were diluted with PBS to a same concentration according to calculation results; and a protein sample and a loading buffer (5×) were thoroughly mixed in a volume ratio of 4:1 and then boiled in an iron bath at 105° C. for 10 min.
(2) Electrophoresis: According to instructions of a gel preparation kit, a separation gel was prepared as a lower gel and a stacking gel was prepared as an upper gel. After being added, the lower gel (separation gel) was flattened with absolute ethanol, and after the upper gel (stacking gel) was added, a comb was inserted as soon as possible to avoid bubbles in a tank (the presence of bubbles would affect electrophoresis of a sample). A sample was taken out from a refrigerator, thawed, then vortexed, and instantaneously centrifuged (because a small amount of a water vapor was condensed at a tube cap to affect a concentration of the sample). A loading amount was determined based on a protein concentration to be 20 μg/well. A rainbow marker was added as a position reference for a target protein at each of two sides of a sample tank, and a cell protein lysate and an autophagosome were loaded sequentially. The electrophoresis was conducted at room temperature, where concentration was conducted at a constant voltage of 70 mV, and after a target protein entered the separation gel, the electrophoresis was conducted at a constant voltage of 130 mV until the target protein reached a required experimental position.
(3) Transfer using electrotransfer solution: An electrotransfer device (an electrotransfer tank, and ice preparation device) and a reagent (an electrotransfer solution: 1,000 mL of the electrotransfer solution included 100 mL of a 10× electrotransfer solution, 700 mL of pure water, and 200 mL of methanol) were prepared. During transfer, a polyvinylidene fluoride (PVDF) membrane was soaked in methanol for 10 s or more to allow activation, no bubbles should appear between a gel and the PVDF membrane, and the electrotransfer solution could be pre-cooled in ice in advance. In an ice tank, the transfer was conducted at a constant current of 300 mA for 1 h (the time could be adjusted according to a size of a target protein).
(4) Blocking: The PVDF membrane was taken out from an electrotransfer tank and soaked in skimmed milk (5%, 5 g of a milk powder was added per 100 ml of water) to allow blocking for 1 h or more. Milk used for the blocking was recovered, stored in a 4° C. refrigerator, and then used the next day to dilute a secondary antibody.
(5) Primary antibody incubation: An LC3 antibody stock solution was diluted with a primary antibody diluent (1:1,000, that is, 1 mL of the primary antibody diluent was added per 1 μL of the primary antibody stock solution), and then the blocked PVDF membrane was incubated in a diluted primary antibody solution overnight at 4° C. under slow shaking on a shaker.
(6) Secondary antibody incubation: The primary antibody was recovered, and then the PVDF membrane was rinsed with PBST (PBS+0.1% Tween) or TBST (tris-buffered saline (TBS) +0.1% Tween), quickly shaken on a shaker 3 times for 10 min each time, and incubated in a diluted secondary antibody solution (a secondary antibody was diluted with a 5% skimmed milk in a ratio of 1:10,000) for 1 h at room temperature.
(7) Exposure: A luminescent solution was prepared, a band was placed in an exposure machine (GE), the luminescent solution was added dropwise on the band for exposure, and an image was acquired. If necessary, the image j software could be used to analyze a gray value of a protein band.

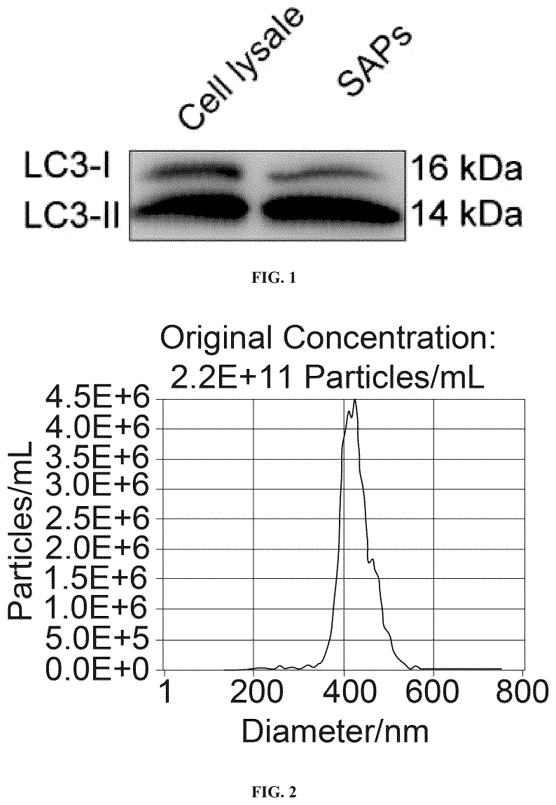

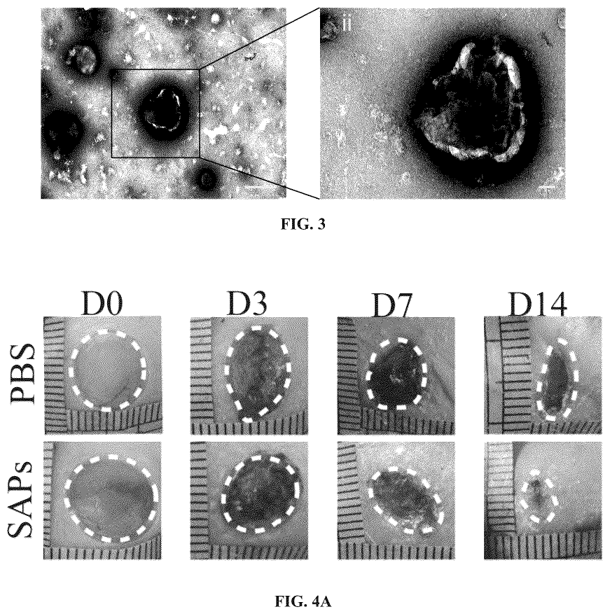

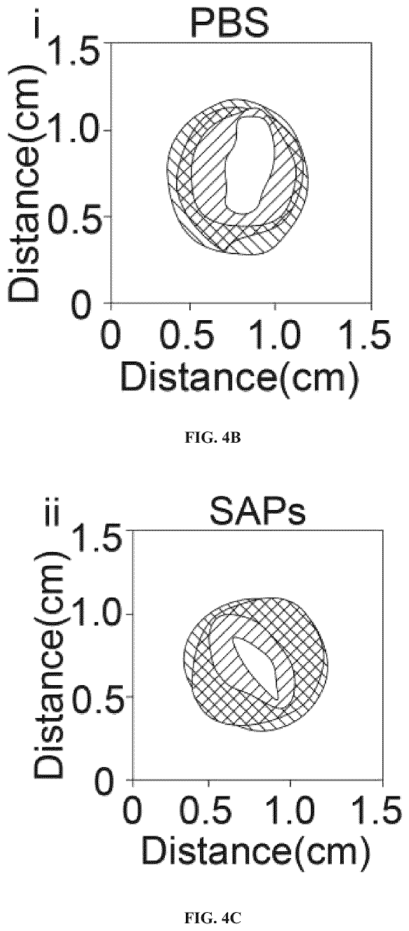

Results are shown in FIG. 1. It can be seen that an expression level of LC3II in the purified autophagosome is significantly higher than an expression level of LC3II in the cell lysate, while an expression level of LC3I in the purified autophagosome is significantly reduced, indicating that the extracted EV is an exosome.
2. A concentration and a particle size of an autophagosome were detected through nanoparticle analysis. A specific detection method was as follows: 20 μL of an autophagosome suspension obtained after centrifugation was taken, diluted with an appropriate amount of PBS, and then placed in a sample chamber of a nanosystem (ParticleMetrix) to allow detection, then result data were processed and analyzed with the software ZetaView 8.04.02, and a particle size and a particle concentration of autophagosomes in each group were recorded. Results are shown in FIG. 2. It can be seen that the two groups of autophagosomes have a similar particle size (415.8±69 nm) and concentration (2.2×10¹¹), indicating that there is no significant difference between the two groups.
3. A morphology of an exosome was detected and observed by transmission electron microscopy, and photographed and recorded. A specific detection method was as follows:
(1) Osmic acid fixation: 20 μL of an autophagosome-containing suspension was added to 500 μL of an osmic acid solution (2%), and a resulting mixed solution was allowed to stand in a 4° C. refrigerator for 2 h to allow fixation.
(2) Dehydration: After the fixation was completed, autophagosomes were washed with PBS 3 times, where a washing system each time was allowed to stand for about 15 min. Washed autophagosomes were subjected to dehydration in ethanol solutions with concentrations of 50%, 70%, 80%, and 90% sequentially, then allowed to further stand for 15 min, and finally soaked in absolute ethanol for 20 min to allow further dehydration. The above dehydration process was repeated once.
(3) Replacement: A sample obtained after the dehydration was soaked in an acetone solution for 15 min to replace the dehydration solution. The above replacement process was repeated once.
(4) Impregnation: A first impregnation solution was prepared with acetone and an embedding agent in a ratio of 2:1, a second impregnation solution was prepared with acetone and an embedding agent in a ratio of 1:1, a third impregnation solution was prepared with acetone and an embedding agent in a ratio of 1:2, and a fourth impregnation solution was prepared with 100% of an embedding agent. A sample obtained after the replacement was impregnated for 2 h in each of the first impregnation solution, the second impregnation solution, and the third impregnation solution sequentially, and finally impregnated for 24 h in the fourth impregnation solution twice.
(5) Embedding: A sample obtained after the impregnation was placed in an embedding mold with a pure embedding agent and subjected to embedding.
(6) Polymerization and sectioning: A sample obtained after the embedding was subjected to polymerization at 65° C. for 48 h, and then sectioned into thin sections each with a thickness of 60 nm to 80 nm.
(7) Staining: The thin sections each were stained in uranyl acetate for 10 min, washed, then stained with lead acetate for 10 min, washed once, and observed under an electron microscope.
(8) Photographing: Each stained section was photographed under an electron microscope (Hitachi) at 100 kV, where an accelerating voltage was set to 40 kV to 120 kV (with an increment of 100 V). Experimental results are shown in FIG. 3. It can be seen that the autophagosomes have a classic double-layer membrane-like structure and are in a cup-like shape overall.

### EXAMPLE 3

In this example, after the relevant verifications were completed and indicated a qualified product based on the method in Example 1 in Example 2, an autophagosome preparation was used to conduct a diabetic mouse treatment test, and specific steps were as follows:

### 1. Establishment of Diabetic Wound Models

20 12-week-old male diabetic mice (db/db mice) with similar sex, age, body weight, and growth conditions were selected and randomly divided into two groups with 10 mice in each group. The mice each were anesthetized, then depilated at a back, and disinfected with a drape, and a wound with a diameter of 1 cm was created on a back of each mouse in a sterile environment.

### 2. Treatment of an Autophagosome on Healing of a Diabetic Wound

A PBS control group and an autophagosome group were set in this experiment. After a wound model of each group was established, in the autophagosome group, an autophagosome suspension was injected at 3, 6, 9, and 12 points around a wound margin every other day, where 3.75 mM of the autophagosome suspension was injected at each point. Mice in the PBS control group each were injected with sterile PBS, where an injection manner and volume were the same as those of the autophagosome group.

### 3. Evaluation of Healing of a Wound and Efficacy of an Autophagosome

### (1) Evaluation of Healing of a Wound

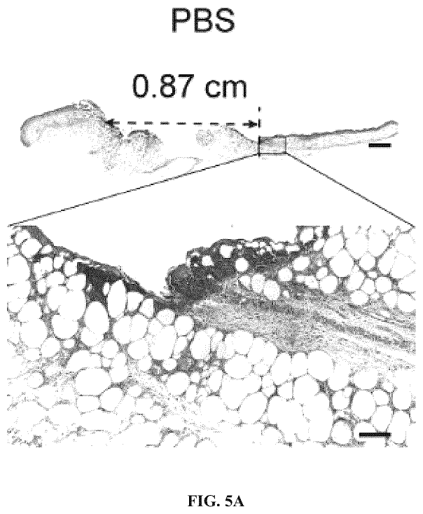

A wound of each of mice in the two groups was photographed, a daily wound healing status was recorded, and a broken rubber ring was replaced. A healing rate of a wound was calculated as follows: (A₀−Aₙ)/A₀×100%, where A₀ represents a wound area on the day of modeling and Aₙ represents a wound area on day n after modeling. An area was calculated by the ImageJ software, and healing rates of wounds were statistically analyzed and plotted after the experiment was completed. On day 3, day 7, and day 14 after wound models were constructed, 6 mice were randomly selected from each of 3 groups and euthanized, and a wound skin was completely cut off (deep to a muscle layer) along a wound margin (about 1 cm away from the wound margin) by surgical scissors and then fixed in a 4% paraformaldehyde fixation solution for subsequent HE staining analysis. Results are shown in FIGS. 4A-C, and it can be seen that the addition of the exosome can significantly shorten a cycle of healing of a wound and improve a repair process of a wound.

### (2) Evaluation of HE Staining

A. Tissue dehydration: A wound tissue block was taken out from the fixation solution, rinsed with running water for 30 min, subjected to dehydration in 75% ethanol, 85% ethanol, 95% ethanol, and absolute ethanol sequentially, and finally placed in a xylene solution to allow alcohol removal.

B. Paraffin embedding and sectioning: A tissue block was placed in melted paraffin with a wound facing upwards to allow embedding, and after the paraffin was cooled and solidified, the tissue block was sectioned by a microtome into thin sections each with a thickness of 5 μm. A thin section was gently tiled on warm water, a glass slide was inserted under a water surface on which the thin section was located, the glass slide was gently lifted, the thin section was flatly attached to the glass slide, and the glass slide was marked with a pencil.

C. Dewaxing: A paraffin section was baked in a baking machine for 2 h to soften paraffin, then statically soaked for 10 min at room temperature in each of a first xylene solution and a second xylene solution sequentially, then statically soaked for 2 min in each of absolute ethanol, 95% ethanol, 85% ethanol, and 75% ethanol sequentially, and finally soaked in water for 2 min.

D. Staining: A dewaxed section was soaked in a hematoxylin solution for 20 min, washed with pure water 3 times, differentiated in 1% hydrochloric acid-ethanol for 10 s, dipped in a 5% ammonia solution for 3 s to allow bluing, then washed with pure water 3 times, statically soaked for 5 min at room temperature in an eosin staining solution, and then rinsed with pure water 3 times.

E. Dehydration, permeabilization, and mounting: A tissue section was subjected to dehydration with ethanol from a low concentration to a high concentration sequentially, and then soaked in xylene continuously for 2 min to make the tissue section permeabilized; and an appropriate amount of a neutral gum was added dropwise on the tissue section on a glass slide, a cover slide was pressed on the glass slide to allow mounting, and the tissue section was observed, photographed, and analyzed with a microscope (Olympus).

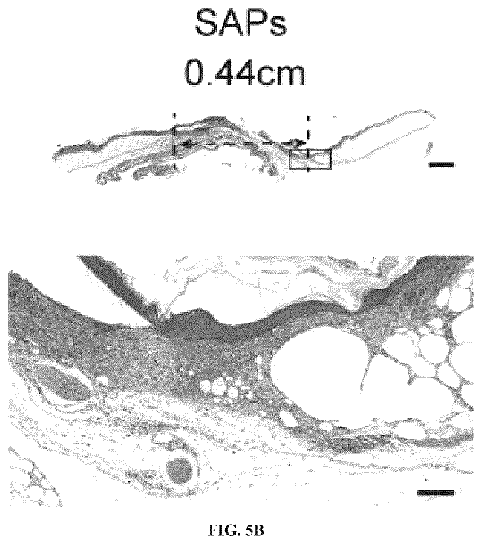

Results are shown in FIGS. 4A-C. After the autophagosome is applied to a diabetic wound, a difference in a size of a wound can be observed on day 7. On day 7, a wound area of the autophagosome group is smaller than a wound area of the control group, where a wound proportion of the control group is 73.5±4.1%, and a wound proportion of the autophagosome group is 45.3±2.5%. On day 14, a wound healing rate of the control group is 49.3±1.7%, and a wound healing rate of the autophagosome group is as high as 90.4±1.4%. The above results show that the autophagosome group can significantly shorten a healing cycle of a wound. A collected wound tissue was subjected to HE staining. Experimental results show that the autophagosome group can significantly promote the epithelization and granulation tissue regeneration of a diabetic wound compared with the control group, but in the control group, no obvious epithelization and granulation tissue can be observed and a large number of adipocytes are filled at a lower edge of a wound (FIGS. 5A-B).

Although the present disclosure has been described in detail through the above examples, the examples are merely some rather than all of the examples of the present disclosure. All other examples obtained by a person based on these examples without creative efforts shall fall within the protection scope of the present disclosure.

What is claimed is:

1. A preparation method of an autophagosome for promoting healing of a diabetic wound, comprising the following steps: cultivating a vascular endothelial cell in a serum-free medium; subjecting a resulting cell culture to first centrifugation to obtain a first supernatant, and collecting the first supernatant; and subjecting the first supernatant to second centrifugation to obtain a precipitate comprising the autophagosome,
wherein the first centrifugation is conducted at a centrifugal force of 2,000 g for 10 min; and
the second centrifugation is conducted at a centrifugal force of 12,000 g for 15 min.

2. The preparation method according to claim 1, wherein the serum-free medium comprises a 0.1% penicillin-streptomycin (PS)-containing Gibco high-glucose Dulbecco's Modified Eagle Medium (DMEM).

3. The preparation method according to claim 2, wherein the vascular endothelial cell is cultivated in the serum-free medium for 48 h.

4. The preparation method according to claim 1, wherein the first centrifugation and the second centrifugation both are conducted at 4° C.

5. The preparation method according to claim 1, wherein after the precipitate is collected, the preparation method further comprises incubating the precipitate with an LC3-labeled magnetic bead to capture the autophagosome.

6. An autophagosome prepared by the preparation method according to claim 1.

7. An autophagosome prepared by the preparation method according to claim 2.

8. An autophagosome prepared by the preparation method according to claim 3.

9. An autophagosome prepared by the preparation method according to claim 4.

10. An autophagosome prepared by the preparation method according to claim 5.

11. A method of use of the autophagosome according to claim 6 in preparation of a drug for promoting healing of a diabetic wound.

12. A method of use of the autophagosome according to claim 7 in preparation of a drug for promoting healing of a diabetic wound.

13. A method of use of the autophagosome according to claim 8 in preparation of a drug for promoting healing of a diabetic wound.

14. A method of use of the autophagosome according to claim 9 in preparation of a drug for promoting healing of a diabetic wound.

15. A method of use of the autophagosome according to claim 10 in preparation of a drug for promoting healing of a diabetic wound.
